(19) United States
(12) Patent Application Publication
(10) Pub. No.: US 2002/0186347 A1
(43) Pub. Date: Dec. 12, 2002

(54) Image displaying apparatus

(75) Inventors: Tatsuki Okamoto, Tokyo (JP); Junichi Nishimae, Tokyo (JP); Yukio Sato, Tokyo (JP)

Correspondence Address: LEYDIG VOIT & MAYER, LTD, 700 THIRTEENTH ST. NW, SUITE 300, WASHINGTON, DC, 20005-3960, US

(73) Assignee: Mitsubishi Denki Kabushiki Kaisha, Tokyo (JP)

(21) Appl. No.: 10/160,115

(22) Filed: Jun. 4, 2002

(30) Foreign Application Priority Data
Jun. 8, 2001 (JP) 2001-173986

(51) Int. Cl. A61B 3/10

(52) U.S. Cl. 351/211

(57) ABSTRACT

An image displaying apparatus for projecting an image of an image plate on the retina of an eyeball. The image displaying apparatus includes a light source, an image plate for transmitting light from the light source, and a lens for converging the light transmitted through the image plate onto an eyeball. A flux of the light passing through the lens is converged into a size approximately the same as that of the pupil of the eyeball, and then, the flux of the light passes through the pupil, and impinges upon the eyeball.

### CROSS-REFERENCE TO RELATED APPLICATIONS

A related patent application is a commonly assigned Japanese Patent Application No. 2001-173986 filed on Jun. 8, 2001, which is incorporated by reference into the present patent application.

### BACKGROUND OF THE INVENTION

1. Field of the Invention

The present invention relates to an image displaying apparatus for projecting an image of an image plate on the retina of an eyeball, and more particularly, to such a wearable image displaying apparatus.

2. Description of the Related Art

FIG. 7 is a structure drawing of an optical system of a conventional image displaying apparatus of the projection-on-eyeball type, indicated in its entirely as 200 (JP, 2-136818, A). Generally used as the image displaying apparatus 200 is an apparatus of the mounted-on-eyeglasses type mounted to eyeglasses or an apparatus of the head mount type mounted to a head.

The image displaying apparatus 200 includes a point light source 201, a liquid crystal panel 202 and an eyepiece 203. After transmitting through the liquid crystal panel 202, light 204 from the point light source 201 is converged by the eyepiece 203 and then impinges upon a human eyeball 205. The light 204 onto the eyeball 205 is focused as an image on a retina 207 through a lens eye 206 from a pupil 208. As the width of the light 204, the range of light (flux of light) irradiating the liquid crystal panel 202 is shown, which is included in light spreading around an optical axis 209.

However, the image displaying apparatus 200 has a problem that since a flux of the light 204 from the point light source 201 is converged at one point on the lens eye 206 inside the pupil 208, the eyeball 205 is subjected to a burden when the image displaying apparatus 200 is used over a long period of time.

Noting this, the inventors of the present invention also studied an image displaying apparatus used with a plane-like light source having about the same size as the liquid crystal panel 202 (not shown). In such an image displaying apparatus, although a burden upon the eyeball 205 is reduced since light is irradiated widely upon the eyeball, a complex optical system for aberration correction is necessary for the purpose of focusing a sharp image on the retina 207. In addition, since the light 204 is incident upon a wider area than the pupil 208, the light 204 always remains incident upon the pupil 208 of an observer even when the observer changes the position of his or her pupil in an attempt to observe a situation in the outer world, and therefore, it is difficult to observe an appearance.

### SUMMARY OF THE INVENTION

An object of the present invention is to provide an image displaying apparatus having a relatively simple optical system and imposing less burden upon an eyeball.

A further object of the present invention is to provide an image displaying apparatus allowing to observe a situation in the outer world while in use.

The present invention is directed to an image displaying apparatus for projecting an image of an image plate on the retina of an eyeball. The image displaying apparatus includes a light source, an image plate for transmitting light from the light source, and a lens for converging the light transmitted through the image plate onto an eyeball. A flux of the light passing through the lens is converged into a size approximately the same as that of the pupil of the eyeball. And then, the flux of the light passes through the pupil, and impinges upon the eyeball.

The present invention is also directed to an image displaying apparatus for projecting an image of an image plate on the retina of an eyeball. The image displaying apparatus includes a light source, an image plate for reflecting light from the light source, and an eyepiece for converging the light reflected by the image plate onto an eyeball. A flux of the light passing through the eyepiece is converged into a size approximately the same as that of the pupil of the eyeball. And then, flux of the light passes through the pupil, and impinges upon the eyeball.

### BRIEF DESCRIPTION OF THE DRAWINGS

FIG. 1 shows the optical system of the image displaying apparatus according to the first preferred embodiment;

FIG. 2 shows the optical system of the image displaying apparatus according to the second preferred embodiment;

FIG. 3 shows the optical system of the image displaying apparatus according to the third preferred embodiment;

FIG. 4 shows the optical system of the image displaying apparatus according to the fourth preferred embodiment;

FIG. 5 shows the optical system of the image displaying apparatus according to the fifth preferred embodiment;

FIG. 6 shows a relationship between the angle of expansion of a beam from a light source and the light intensity; and

FIG. 7 shows the optical system of the conventional image displaying apparatus.

### DETAILED DESCRIPTION OF THE PREFERRED EMBODIMENTS

First Preferred Embodiment

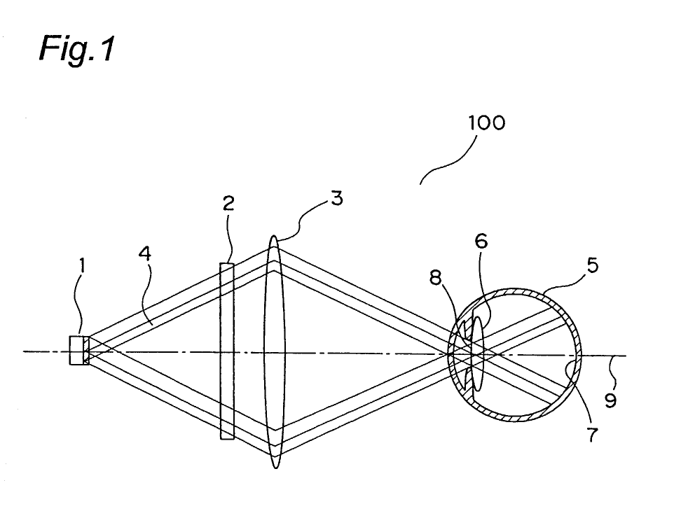

FIG. 1 shows an optical system of an image displaying apparatus of the projection-on-eyeball type according to a first preferred embodiment, indicated in its entirely as 100.

The image displaying apparatus 100 includes a light source 1, a liquid crystal panel 2 and an eyepiece 3. The light source 1 is an aggregation of more than one point light sources. In FIG. 1 for example, three point light sources are arranged side by side in the vertical direction. Light 4 from the light source 1 impinges upon an image plate 2 while expanding around an optical axis 9. For the image plate 2, a liquid crystal panel or film plate is used, for instance. The light 4, transmitted through the image plate 2, is converged by the eyepiece 3 onto an eyeball 5 of an observer.

The light 4 incident upon the eyeball 5 is focused on a retina 7 through a lens eye 6 from a pupil 8 for image formation. Hence, the observer visually recognizes the image displayed on the image plate 2.

In the image displaying apparatus 100 according to the first preferred embodiment, the size of the light source 1, a focal distance of the eyepiece 3, a distance between the light source 1 and the eyepiece 3, a distance between the eyepiece 3 and the pupil 8 are adjusted, whereby a flux of the light 4 passing through the eyepiece 3 is converged into approximately the same size as that of the pupil 8 and accordingly passes through the pupil.

Since the light 4 expands approximately equally around the optical axis 9, it is preferable that the flux of the light 4 at the moment of passing through the pupil 8 has an approximately circular cross section of approximately the same size as that of the pupil 8 in diameter in a direction approximately perpendicular to the optical axis 9.

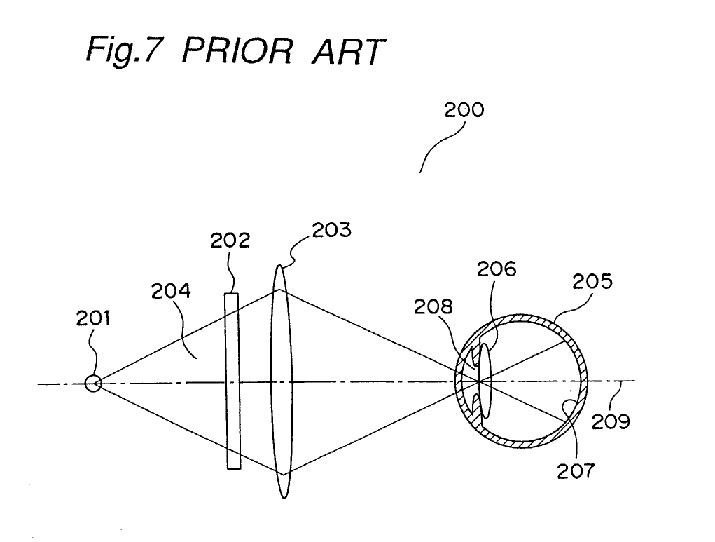

Thus, in the image displaying apparatus 100, since the light 4 does not converge to one point in the lens eye 6 as it does in the case of the conventional image displaying apparatus 200 (See FIG. 7.), even when the image displaying apparatus 100 is used over a long period of time, almost no burden is imposed upon the eyeball 5.

From a condition of watching an image on the image plate 2, even a slight move of the eyeball 5 inhibits a part or all of the light 4 from passing through the pupil 8. Hence, merely rolling the eyeball 5 just slightly, the observer can easily observe a situation in the outer world. A distance for the move of the eyeball 5 by the observer is preferably about half the diameter of the pupil 8 or larger.

This is useful when the image displaying apparatus 100 according to the first preferred embodiment is used as an image displaying apparatus of the mounted-on-eyeglasses type or the head mount type. That is, this is very useful when an attention to a situation in the outer world must be paid while wearing the image displaying apparatus 100 is required, for instance, in case of walking outside a building while wearing the image displaying apparatus, or obtaining information from the image displaying apparatus if necessity while looking at a situation in the outer world, or on other occasions.

Second Preferred Embodiment

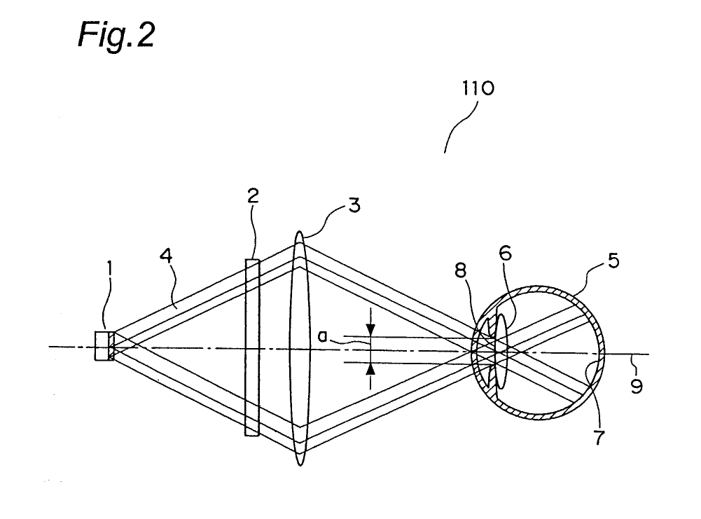

FIG. 2 shows an optical system of an image displaying apparatus according to a second preferred embodiment, indicated in its entirely as 110. In FIG. 2, the same reference symbols as those used in FIG. 1 denote the same or corresponding portions.

In the image displaying apparatus 110, the size of the light source 1 or the like are adjusted in such a manner that a flux of the light 4 passing through the pupil 8 has an approximately circular cross section of a predetermined diameter a in a direction which is approximately perpendicular to the optical axis 9.

The diameter a is preferably 2 mm or larger but 7 mm or smaller, and more preferably, 2 mm or larger but 4 mm or smaller.

When using the image displaying apparatus 110, as an observer moves his or her eyeball 5 just about 3.5 mm, or preferably about 2 mm, from a condition of turning his or her eye on the image plate 2 and observing an image, the light 4 into the eyeball 5 can be blocked. Hence, clear observation of a situation in the outer world is realized, with the image on the image plate 2 not coming onto the eyeball 5.

Third Preferred Embodiment

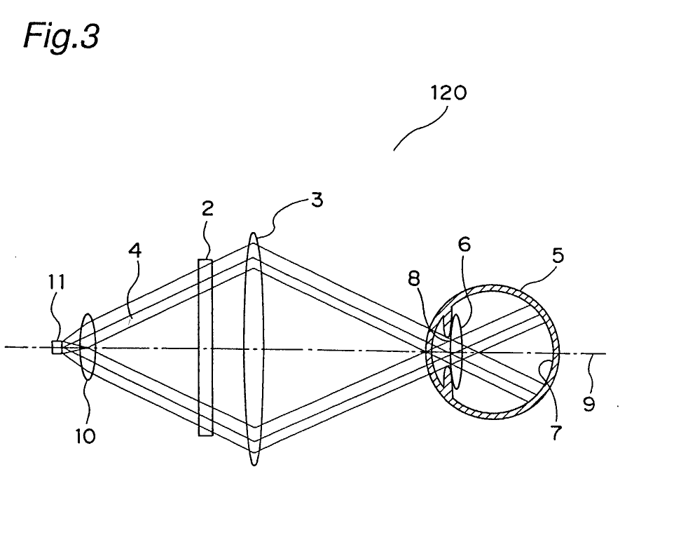

FIG. 3 shows an optical system of an image displaying apparatus according to a third preferred embodiment, indicated in its entirety as 120. In FIG. 3, the same reference symbols as those used in FIG. 1 denote the same or corresponding portions.

In the image displaying apparatus 120, a white light emitting diode (hereinafter referred to as “LED”) 11 is used as a light source, and there is a lens 10 disposed between the LED 11 and the image plate 2.

As the LED 11 is used as the light source as described above, the high optical directivity of the LED 11 promises the following two effects in addition to the effects according to the first and the second preferred embodiments described above. That is, first, use of even a relatively simple optical system allows an observer to recognize a clear image. Second, since the spreading of the light from the light source is narrow, the light emitted from the light source can be utilized effectively.

Depending on the optical system, the lens 10 may not be used.

Fourth Preferred Embodiment

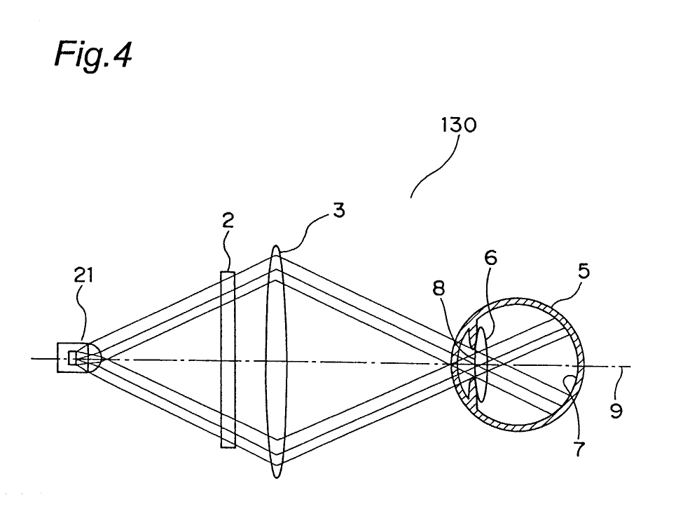

FIG. 4 shows an optical system of an image displaying apparatus according to a fourth preferred embodiment, indicated in its entirety as 130. In FIG. 4, the same reference symbols as those used in FIG. 1 denote the same or corresponding portions.

In the image displaying apparatus 130, a white light emitting diode with lens (hereinafter referred to as “LED with lens”) 21 is used as a light source.

As the LED with lens 21 is used as the light source as described above, it is possible to simplify the optical system of the image displaying apparatus, reduce the number of components and parts, and provide the image displaying apparatus more inexpensively, in addition to the effects according to the third preferred embodiment described above.

Fifth Preferred Embodiment

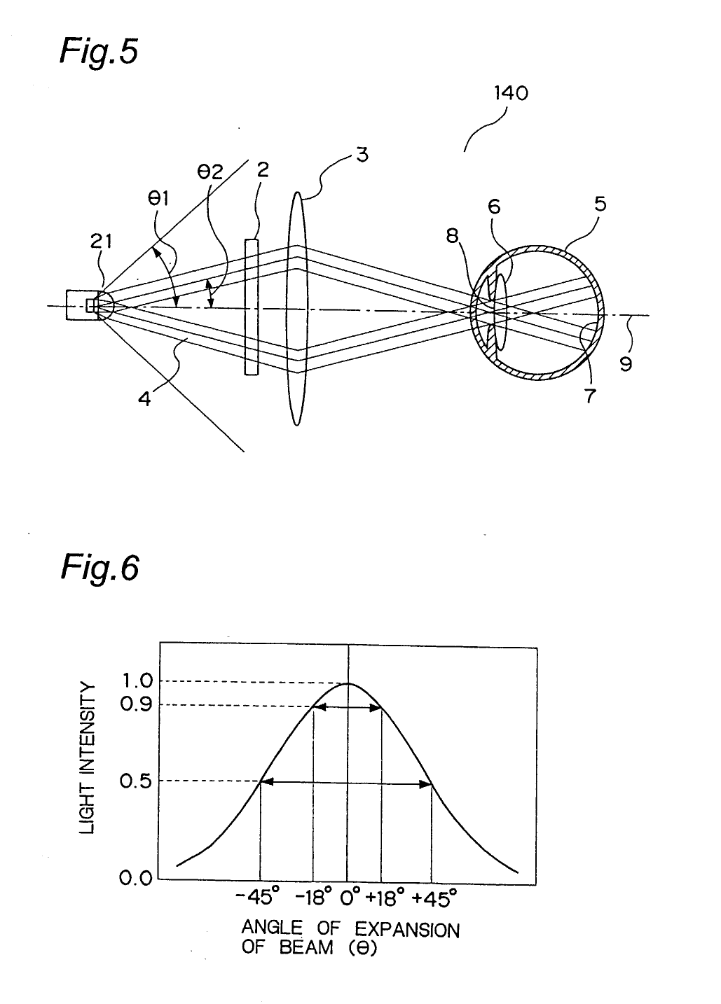

FIG. 5 shows an optical system of an image displaying apparatus according to a fifth preferred embodiment, indicated in its entirety as 140. In FIG. 5, the same reference symbols as those used in FIG. 4 denote the same or corresponding portions.

Denoted by θ1 is the angle of divergence of the LED with lens 21. The angle of divergence θ1 is expressed as an angle measured from the optical axis 9 to a position where the light intensity becomes half that on the optical axis 9. Denoted by θ2 is the angle of inclination from the optical axis. In the fifth preferred embodiment, only light with θ2 of 18 degrees or smaller is incident upon an image displaying region included in the image plate. It is particularly preferable that θ2 is set about 10 degrees.

FIG. 6 shows a relationship between the light intensity emitted from the LED with lens 21 and the angle of expansion (θ) of a beam (ray) measured from the optical axis 9. The light intensity is expressed by a proportion to the light intensity on the optical axis 9 assuming that the light intensity on the optical axis 9 is 1.

As shown in FIG. 6, when a light source (LED with lens 21) having the angle of divergence (the light intensity becomes half that on the optical axis at the angle) of 45 degrees is used, if the angle of inclination θ2 is 18 degrees, it is possible to ensure that only light with light intensity of about 0.9 times as large as the light intensity on the optical axis 9 or stronger impinges upon the image displaying region included in the image plate.

As a result, it is possible to make an image recognized by an observer brighter and reduce a distribution of the brightness, in addition to the effects according to the fourth preferred embodiment described above.

Although the eyepiece 3 is formed by one lens in the first through the fifth preferred embodiments, the eyepiece 3 may be formed as a combination of a plurality of lenses.

In addition, while the foregoing has described that a liquid crystal panel of the transmission type or the like is used as the image plate 2, a liquid crystal panel of the reflection type or the like may be used. In that case, light from the light source is reflected at the surface of the image plate, and then converged by the eyepiece 3 to impinge upon the eyeball 5.

According to the first through the fifth preferred embodiments, an image displaying apparatus allowing to clearly observe a situation in the outer world while wearing the image displaying apparatus is provided.

Also, an image displaying apparatus permitting to see a clear image is provided using a relatively simple optical system.

Furthermore, an image displaying apparatus permitting to see a bright image with brightness of uniform is provided.

What is claimed is:

1. An image displaying apparatus for projecting an image of an image plate on the retina of an eyeball, comprising:
a light source;
an image plate for transmitting light from said light source; and
a lens for converging said light transmitted through said image plate onto an eyeball,
characterized in that a flux of said light passing through said lens is converged into a size approximately the same as that of the pupil of said eyeball, passes through said pupil, and impinges upon said eyeball.

2. An image displaying apparatus according to claim 1, characterized in that the flux of said light at the moment of passing through said pupil has an approximately circular cross section of approximately the same size as that of said pupil in diameter in a direction approximately perpendicular to the optical axis of said light.

3. An image displaying apparatus according to claim 2, characterized in that the diameter of said approximately circular cross section is 2 mm or larger but 7 mm or smaller.

4. An image displaying apparatus according to claim 2, characterized in that the diameter of said approximately circular cross section is 2 mm or larger but 4 mm or smaller.

5. An image displaying apparatus according to claim 1, further comprising a lens disposed between said light source and said image plate for transmitting said light.

6. An image displaying apparatus according to claim 1, characterized in that said light source is a light emitting diode.

7. An image displaying apparatus according to claim 6, characterized in that said light emitting diode is a white light emitting diode.

8. An image displaying apparatus according to claim 1, characterized in that said light source, is a light emitting diode with lens obtained by integrating a light emitting diode and a lens for transmitting light emitted from said light emitting diode.

9. An image displaying apparatus according to claim 1, characterized in that the angle of divergence of outgoing light emitted from said light source is 45 degrees or larger, and said outgoing light at an angle of inclination of 18 degrees or smaller with respect to the optical axis impinges upon an image displaying region included in said image plate.

10. An image displaying apparatus according to claim 9, characterized in that the angle of inclination is about 10 degrees.

11. An image displaying apparatus for projecting an image of an image plate on the retina of an eyeball, comprising:
a light source;
an image plate for reflecting light from said light source; and
an eyepiece for converging said light reflected by said image plate onto an eyeball, characterized in that a flux of said light passing through said eyepiece is converged into a size approximately the same as that of the pupil of said eyeball, passes through said pupil, and impinges upon said eyeball.

12. An image displaying apparatus according to claim 11, characterized in that the flux of said light at the moment of passing through said pupil has an approximately circular cross section of approximately the same size as that of said pupil in diameter in a direction approximately perpendicular to the optical axis of said light.

13. An image displaying apparatus according to claim 12, characterized in that the diameter of said approximately circular cross section is 2 mm or larger but 7 mm or smaller.

14. An image displaying apparatus according to claim 12, characterized in that the diameter of said approximately circular cross section is 2 mm or larger but 4 mm or smaller.

15. An image displaying apparatus according to claim 11, further comprising a lens disposed between said light source and said image plate for transmitting said light.

16. An image displaying apparatus according to claim 11, characterized in that said light source is a light emitting diode.

17. An image displaying apparatus according to claim 16, characterized in that said light emitting diode is a white light emitting diode.

18. An image displaying apparatus according to claim 11, characterized in that said light source is a light emitting diode with lens obtained by integrating a light emitting diode and a lens for transmitting light emitted from said light emitting diode.
